SEQUENCE LISTING (46 sequences)

SEQ ID NO: 1 - DNA, 237 bp, Homo sapiens
  misc_feature 1..237: note = FAF1 Human
  source 1..237: mol_type = other DNA
aatgctgagcctgtgagcaaactgcggatccggacccccagtggcgagttcttggagcggcgtttcctggccagcaacaagctccagattgtctttgattttgtagcttccaaaggatttccatgggatgagtacaagttactgagcacctttcctaggagagacgtaactcaactggacccaaataaatcattattggaggtaaagttgttccctcaagaaacccttttccttgaa

SEQ ID NO: 2 - protein, 78 aa, Homo sapiens
  REGION 1..78: note = FAF1 Human
NAEPVSKLRIRTPSGEFLERRFLASNKLQIVFDFVASKGFPWDEYKLLSTFPRRDVTQLDPNKSLLEVKLFPQETLFL

SEQ ID NO: 3 - DNA, 690 bp, synthetic construct
  misc_feature 1..690: note = p97-PROTAC (with an anti-GFP nanobody)
  source 1..690: mol_type = other DNA
atggaacaaaaactcatctcagaagaggattctagaatggatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtctccagcactagtgaattcaaagaaagcggcagcgtgagcagcgaacagctggcgcagtttcgcagcctggataatgcagaaccagtgtctaaactcagaataaggaccccaagcggtgagttcctcgagcggcgcttcttggcctctaacaaacttcaaattgtattcgattttgttgcctctaagggttttccttgggacgaatacaaactgctttctacgttccccagaagggatgttacccaactggacccgaataaatcactgcttgaggttaagttgtttccgcaggaaacattgtttctttaa

SEQ ID NO: 4 - protein, 229 aa, synthetic construct
  REGION 1..229: note = p97-PROTAC (with an anti-GFP nanobody)
MEQKLISEEDSRMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSTSEFKESGSVSSEQLAQFRSLDNAEPVSKLRIRTPSGEFLERRFLASNKLQIVFDFVASKGFPWDEYKLLSTFPRRDVTQLDPNKSLLEVKLFPQETLFL

SEQ ID NO: 5 - DNA, 750 bp, synthetic construct
  misc_feature 1..750: note = p97-PROTAC (with an anti-alpha synuclein nanobody, Nb87)
  source 1..750: mol_type = other DNA
atggaacaaaaactcatctcagaagaggattctagaatgcaggtacaacttcaagagtcaggaggtggttcagtacaaaccggcggtagtttacgcctgtcatgcgtagcgtcaggatatagcggatacatggcatggtttagacaagcacctggtaaagaaagagagggcattgcggcaatatatcggggagacaaaattacctactatgcccattctgtccagggtcgctttaccatctcccaggccaacgcaaaaaacaccgtgtatcttttaatgaactcattgaagccggaagacacggctatttactactgtgctgcgagaagagtcgttgctgactcaccattgttgtcaaaaacgtatgcttattgggggcagggaacccaagtgaccgtatctagtggggaacaaaaactcatctcagaagaggatctgtctagatctctcgaggaattcaaagaaagcggcagcgtgagcagcgaacagctggcgcagtttcgcagcctggataatgcagaaccagtgtctaaactcagaataaggaccccaagcggtgagttcctcgagcggcgcttcttggcctctaacaaacttcaaattgtattcgattttgttgcctctaagggttttccttgggacgaatacaaactgctttctacgttccccagaagggatgttacccaactggacccgaataaatcactgcttgaggttaagttgtttccgcaggaaacattgtttctttaa

SEQ ID NO: 6 - protein, 249 aa, synthetic construct
  REGION 1..249: note = p97-PROTAC (with an anti-alpha synuclein nanobody, Nb87)
MEQKLISEEDSRMQVQLQESGGGSVQTGGSLRLSCVASGYSGYMAWFRQAPGKEREGIAAIYRGDKITYYAHSVQGRFTISQANAKNTVYLLMNSLKPEDTAIYYCAARRVVADSPLLSKTYAYWGQGTQVTVSSGEQKLISEEDLSRSLEEFKESGSVSSEQLAQFRSLDNAEPVSKLRIRTPSGEFLERRFLASNKLQIVFDFVASKGFPWDEYKLLSTFPRRDVTQLDPNKSLLEVKLFPQETLFL

SEQ ID NO: 7 - DNA, 297 bp, Homo sapiens
  misc_feature 1..297: note = UBXN1 human
  source 1..297: mol_type = other DNA
cgggagtatgaccagtgtcgcatacaggtcaggctgccagatgggacctcactgacccagacgttccgggcccgggaacagctggcagctgtgaggctctatgtggagctccaccgtggggaggaactaggtgggggccaggaccctgtgcaattgctcagtggcttccccagacgggccttctcagaagctgacatggagcggcctctgcaggagctgggtatggctgcaagactagaaaccaggactagaaactgggggagtagggaggcatgcctaggaaaaggagggatgcaa

SEQ ID NO: 8 - protein, 83 aa, Homo sapiens
  REGION 1..83: note = UBXN1 Human
REYDQCRIQVRLPDGTSLTQTFRAREQLAAVRLYVELHRGEELGGGQDPVQLLSGFPRRAFSEADMERPLQELGLVPSAVLIV

SEQ ID NO: 9 - DNA, 261 bp, Homo sapiens
  misc_feature 1..261: note = UBXN2A Human
  source 1..261: mol_type = unassigned DNA
aacttggaacccattactaatatacagatctggttggccaatggaaaaaggattgtccagaaatttaacattactcatagagtaagccatatcaaagacttcattgaaaaataccaaggatctcaaagaagtcctccgttttccctggcaacagctcttcctgtcctcaggttgctagatgagacactcacactggaagaagcagatttacagaatgctgtcatcattcagagactccaaaaaactgcatcttttagagaa

SEQ ID NO: 10 - protein, 86 aa, Homo sapiens
  REGION 1..86: note = UBXN2A
NLEPITNIQIWLANGKRIVQKFNITHRVSHIKDFIEKYQGSQRSPPFSLATALPVLRLLDETLTLEEADLQNAVIIQRLQKTASFR

SEQ ID NO: 11 - DNA, 237 bp, Homo sapiens
  misc_feature 1..237: note = UBXN2B
  source 1..237: mol_type = other DNA
gattcagtgccaacaacaaaaattcaaatcaggttagcagatgggagtcgtttgatacaaagattcaatagtacacacaggatcctggatgtccggaactttattgtacagtctcgtcctgaatttgcggctcttgactttattcttgtgacttcatttccgaataaagagctaacagatgaaagcctgacactgctagaagcagatattcttaacactgtgttactccagcaacta

SEQ ID NO: 12 - protein, 78 aa, Homo sapiens
  REGION 1..78: note = UBXN2B
DSVPTTKIQIRLADGSRLIQRFNSTHRILDVRNFIVQSRPEFAALDFILVTSFPNKELTDESLTLLEADILNTVLLQQ

SEQ ID NO: 13 - DNA, 243 bp, Homo sapiens
  misc_feature 1..243: note = NSFL1C
  source 1..243: mol_type = other DNA
gaatcagagcctaccacaaacatccaaattcggcttgcagacggcgggaggctggtgcagaaatttaaccacagccacaggatcagcgacatccgactcttcatcgtggatgcccggccagccatggctgccaccagctttatcctcatgactactttcccgaacaaagagctggctgatgagagccagaccctgaaggaagccaacctgctcaatgctgtcatcgtgcagcggttaacataa

SEQ ID NO: 14 - protein, 80 aa, Homo sapiens
  REGION 1..80: note = NSFL1C
ESEPTTNIQIRLADGGRLVQKFNHSHRISDIRLFIVDARPAMAATSFILMTTFPNKELADESQTLKEANLLNAVIVQRLT

SEQ ID NO: 15 - DNA, 252 bp, Homo sapiens
  misc_feature 1..252: note = FAF2
  source 1..252: mol_type = other DNA
gatgaccctgaaagtgtcaagatcatcttcaaattacctaatgattctcgagtagagagacgattccacttttcacagtctctaacagtaatccacgacttcttattctccttgaaggaaagcccagaaaagtttcagattgaagccaattttcccaggcgagtgctgccctgcatcccttcagaggagtggcccaatccccctacgctacaggaggccggactcagccacacagaagttctttttgttcag

SEQ ID NO: 16 - protein, 83 aa, Homo sapiens
  REGION 1..83: note = FAF2
DDPESVKIIFKLPNDSRVERRFHFSQSLTVIHDFLFSLKESPEKFQIEANFPRRVLPCIPSEEWPNPPTLQEAGLSHTEVLFV

SEQ ID NO: 17 - DNA, 240 bp, Homo sapiens
  misc_feature 1..240: note = UBXN4
  source 1..240: mol_type = other DNA
gaaagaagcactgttgcaagaattcaattccgtcttcctgatggttcttcctttacaaatcagttcccttctgatgctcctctagaagaggcaaggcagtttgctgcacagactgttggcaacacttacggtaatttttcgttagcaaccatgtttcccaggagggaatttaccaaagaagattataaaaagaagttactggatttggaacttgccccaagcgcttcggtggtactgttg

SEQ ID NO: 18 - protein, 79 aa, Homo sapiens
  REGION 1..79: note = UBXN4
ERSTVARIQFRLPDGSSFTNQFPSDAPLEEARQFAAQTVGNTYGNFSLATMFPRREFTKEDYKKKLLDLELAPSASVVL

SEQ ID NO: 19 - DNA, 331 bp, Homo sapiens
  misc_feature 1..331: note = UBXN6
  source 1..331: mol_type = other DNA
cgcaagtacaactacacgctgctgcgcgtgcgcctccccgatggctgcctcctgcagggcactttctacgctcgggagcggctgggggcggtgtacgggttcgtccgggaggccctgcagagcgactggctgccttttgagctgctggcctcgggagggcagaagctgtccgaggacgagaacctggccttgaacgagtgcgggctggtgccctctgccctcctgaccttctcgtgggacatggctgtgctggaggacatcaaggccgcgggggccgagccggactccatcctgaaacccgagctcctgtcagccatcgagaagctcttgt

SEQ ID NO: 20 - protein, 77 aa, Homo sapiens
  REGION 1..77: note = UBXN6
RKYNYTLLRVRLPDGCLLQGTFYARERLGAVYGFVREALQSDWLPFELLASGGQKLSEDENLALNECGLVPSALLTF

SEQ ID NO: 21 - DNA, 265 bp, Homo sapiens
  misc_feature 1..265: note = UBXN7
  source 1..265: mol_type = other DNA
gtaaatggaccaaaagcacagctgatgttgcggtatccagatggaaaaagggaacagatcactcttccagagcaagctaaactgctagctttggtgaagcacgtgcagtctaaaggatacccaaatgaacgttttgaacttctcaccaactttcctcgaaggaaattatctcatctggactatgatattacattgcaagaggcaggcctttgtcctcaagagactgtctttgtacaggaaagaaattaacaccatggcttgacct

SEQ ID NO: 22 - protein, 78 aa, Homo sapiens
  REGION 1..78: note = UBXN7
VNGPKAQLMLRYPDGKREQITLPEQAKLLALVKHVQSKGYPNERFELLTNFPRRKLSHLDYDITLQEAGLCPQETVFV

SEQ ID NO: 23 - DNA, 234 bp, Homo sapiens
  misc_feature 1..234: note = UBXN8
  source 1..234: mol_type = other DNA
acagcagaagaagtagttactgttgctctccgatgtcccagtgggaatgtcctgaggagaaggtttttgaagtcctacagctcacaggtcttatttgactggatgacgagaattgggtaccacatatctctatacagcctttctacttcctttcccagacggcctctggcagtggagggaggccagtcgctggaggacataggaataactgtggacactgtactcatcctggag

SEQ ID NO: 24 - protein, 77 aa, Homo sapiens
  REGION 1..77: note = BXN8
TAEEVVTVALRCPSGNVLRRRFLKSYSSQVLFDWMTRIGYHISLYSLSTSFPRRPLAVEGGQSLEDIGITVDTVLIL

SEQ ID NO: 25 - DNA, 234 bp, Homo sapiens
  misc_feature 1..234: note = ASPSCR1
  source 1..234: mol_type = other DNA
gagcgctacccaaaggtggctctgagggtcctgttccccgaccgctacgtcctacagggcttcttccgccccagcgagacagtgggggacttgcgagacttcgtgaggagccacctggggaaccccgagctgtcattttacctgttcatcacccctccaaaaacagtcctggacgaccacacgcagaccctctttcaggcgaacctcttcccggccgctctggtgcacttggga

SEQ ID NO: 26 - protein, 77 aa, Homo sapiens
  REGION 1..77: note = ASPSCR1
ERYPKVALRVLFPDRYVLQGFFRPSETVGDLRDFVRSHLGNPELSFYLFITPPKTVLDDHTQTLFQANLFPAALVHL

SEQ ID NO: 27 - DNA, 264 bp, Homo sapiens
  misc_feature 1..264: note = UBXN10
  source 1..264: mol_type = other DNA
gaccaagaaccaaggttgctgcttgctgttagatcaccaacaggccaaaggtttgtacgccatttccggccaacagatgatttgcaaaccattgttgctgtggccgaacagaaaaacaaaacctcctaccgacactgcagcattgaaacaatggaggtgcccaggaggcgattttctgacctcaccaaatctctgcaagagtgcagaatcccccacaagtctgtgctgggcatctcactggaagatggggaagggtggccctga

SEQ ID NO: 28 - protein, 87 aa, Homo sapiens
  REGION 1..87: note = UBXN10
DQEPRLLLAVRSPTGQRFVRHFRPTDDLQTIVAVAEQKNKTSYRHCSIETMEVPRRRFSDLTKSLQECRIPHKSVLGISLEDGEGWP

SEQ ID NO: 29 - DNA, 138 bp, Homo sapiens
  misc_feature 1..138: note = UBXN11
  source 1..138: mol_type = other DNA
gctctgctagcgcaggccagggtcatggatgcctctgcctttgagatcttcagcacattcccgcccaccctctaccaggacgatacactcacgctgcaggctgcaggccttgtgcccaaagcagcactgctgctgcgg

SEQ ID NO: 30 - protein, 78 aa, Homo sapiens
  REGION 1..78: note = UBXN11
PAPPLSMLRIKSENGEQAFLLMMQPDNTIGDVRALLAQARVMDASAFEIFSTFPPTLYQDDTLTLQAAGLVPKAALLL

SEQ ID NO: 31 - DNA, 240 bp, Aedes aegypti
  misc_feature 1..240: note = UBXN1 ortholog Aedes aegypti (Yellowfever mosquito) (Culex aegypti)
  source 1..240: mol_type = other DNA
aagacctacaccaatgctcgcattcaaattcgcatgttggacggcagcaccctggtggagactttcgacgccaaggaacagctcgccgccgtgcgactgttcgtccagcttaaattgggcagcggctcggctccgttcggcttgatgaccagcttcccgcggaaggtgttcgacggagaggacttcgacaactcgttggaaaagttgggactggttccgtcggcggtactaatcgtgact

SEQ ID NO: 32 - protein, 79 aa, Aedes aegypti
KTYTNARIQIRMLDGSTLVETFDAKEQLAAVRLFVQLKLGSGSAPFGLMTSFPRKVFDGEDFDNSLEKLGLVPSAVLIV

SEQ ID NO: 33 - DNA, 243 bp, Aedes aegypti
  misc_feature 1..243: note = NSFL1C ortholog Aedes aegypti (Yellowfever mosquito) (Culex aegypti)
  source 1..243: mol_type = other DNA
gaaagccaaccgactaccaatctgcagattcggctggctgatggatctcgtttgtcggcacgtttcaaccaaagccatacaatcgataacgtgcgccagtatataacaaacgctcgtccccaatacgcatcggcgagtttcgctctgatgacaacattccccccgaaggagcttgccgatggttcccagagcttgaaggatgcgggattgttgaatgcggcgataatgcaacgaatgaagtag

SEQ ID NO: 34 - protein, 80 aa, Aedes aegypti
  REGION 1..80: note = NSFL1C ortholog Aedes aegypti (Yellowfever mosquito) (Culex aegypti)
ESQPTTNLQIRLADGSRLSARFNQSHTIDNVRQYITNARPQYASASFALMTTFPPKELADGSQSLKDAGLLNAAIMQRMK

SEQ ID NO: 35 - DNA, 237 bp, Aedes aegypti
  misc_feature 1..237: note = FAF1 ortholog Aedes aegypti (Yellowfever mosquito) (Culex aegypti)
  source 1..237: mol_type = other DNA
attggagaaaacataacaaaaattagaattaggacaccaggcggctcaatgctggaaagacgcttcacaacggacaccccactgcgcattctgttgaactacatcaccggcgaagggttcctagtagacgagttcaaggtgatttccagctggccgcgaagagacctcacaacgctggactacgagaacacaatgaaggatctgaagctctacccccaggagaccctaatactggag

SEQ ID NO: 36 - protein, 78 aa, Aedes aegypti
  REGION 1..78: note = FAF1 ortholog Aedes aegypti (Yellowfever mosquito) (Culex aegypti)
IGENITKIRIRTPGGSMLERRFTTDTPLRILLNYITGEGFLVDEFKVISSWPRRDLTTLDYENTMKDLKLYPQETLIL

SEQ ID NO: 37 - DNA, 237 bp, Aedes aegypti
  misc_feature 1..237: note = UBXN6 ortholog Aedes aegypti (Yellowfever mosquito) (Culex aegypti)
  source 1..237: mol_type = other DNA
aaccgttacaagtttacgctgataagagttcgctttcctaacggggtttttctgcaaggaaccttcaacgtgtacgaaaagctgtcccaagtgtatgaatttgtacagtcctgcctaatgcatgaagcgtcggaatttgtcctgatctcatctaccgaacagcgattcacggaagccgactttgacaaatcgctgtacgacctaaaattggttcccacgattttgtttaactttagc

SEQ ID NO: 38 - protein, 78 aa, Aedes aegypti
  REGION 1..78: note = UBXN6 ortholog Aedes aegypti (Yellowfever mosquito) (Culex aegypti)
NRYKFTLIRVRFPNGVFLQGTFNVYEKLSQVYEFVQSCLMHEASEFVLISSTEQRFTEADFDKSLYDLKLVPTILFNF

SEQ ID NO: 39 - DNA, 300 bp, Aedes aegypti
  misc_feature 1..300: note = ASPSCR1 ortholog Aedes aegypti (Yellowfever mosquito) (Culex aegypti)
  source 1..300: mol_type = other DNA
gaagaacagcctttgatgacgttcgaactgcgcaagctggaagacgaaaagcgtgtgttgaacaatttggcgcgctaccgaaatacgatcatccgggtccagttccccgaccggtacgtcctgcagggaatcttcaaactacacgagaccattgccgatgtgatagctttctcacaagcgcatctagaggaccaattcgcagcgattcatctttatactactcctccgaagacaatcctatcacccgaacttactttggttgaagcaaaatgtgttccccaagcaatactgcatgtcggc

SEQ ID NO: 40 - protein, 99 aa, Aedes aegypti
  REGION 1..99: note = ASPSCR1 ortholog Aedes aegypti (Yellowfever mosquito) (Culex aegypti)
EEQPLMTFELRKLEDEKRVLNNLARYRNTIIRVQFPDRYVLQGIFKLHETIADVIAFSQAHLEDQFAAIHLYTTPPKTILSPELTLVEAKCVPQAILHV

SEQ ID NO: 41 - DNA, 2721 bp, Homo sapiens
  misc_feature 1..2721: note = VCIP135 Human UBX-Like
  source 1..2721: mol_type = other DNA
gctccacagactccgtcgtccttggcgtcggcggctgcttcgggggggcttttgaagcggagagaccggagaatcctttccgggagctgcccggatccgaagtgtcaggcgcgtctatttttcccggcctccggttctgtcagcatcgagtgtaccgagtgcggccagcggcacgagcagcaacagctgctgggggttgaggaggtgaccgacccggacgtagtgctacacaacctgctgcggaacgcgctgctcggggttacgggggcacccaagaagaacacggaactggtaaaggtgatgggcctttccaactatcactgcaaattgttgtcgcccatattagctcgctatggaatggacaaacagacaggccgggccaagcttctccgggacatgaaccagggcgaactgttcgattgcgccttactgggtgaccgcgccttcctcatagaaccagagcatgttaacactgtgggctatggcaaggaccgctccggaagcctcctgtatttgcatgacactctggaggacattaagcgggccaataaaagccaggaatgtctcattccagtgcatgtggacggggatggacactgcttggtgcatgctgtgtctcgggctctagtaggccgagagctcttctggcatgccttaagagagaatcttaaacagcactttcagcagcacctggcccgatatcaagctctgttccatgacttcattgatgctgctgagtgggaggacattatcaatgagtgtgaccctctgtttgtaccacctgagggtgttcccttgggcctgaggaatatccacatatttggtcttgccaatgtgctacatcgtcctattattctgttagattccctcagtggcatgagaagctctggtgattattcagccacctttctacctgggctcatccctgcagagaagtgcactgggaaagatggtcatttgaacaaaccaatctgtattgcatggagcagctccggtagaaaccattatatccccttggtaggcataaaaggggctgctttgcccaaactgcctatgaatttgcttcctaaagcatggggtgtgcctcaggaccttattaaaaagtacataaaacttgaagaggatggtggttgtgttattggaggtgacagaagtttgcaagataaatacttacttaggcttgttgctgctatggaagaagtctttatggacaaacatggtatccatcctagtttggttgctgatgtccatcagtatttctacagaaggactggagtgataggagttcagcctgaggaagtcacagcagctgctaaaaaagcagtaatggataatcgccttcacaaatgtttgctctgtggtgccctttctgaacttcatgttcctccagagtggttggctcctggagggaaattgtataacctggcaaaaagtactcatggacagctgaggactgacaaaaattacagctttcccttgaacaatttggtttgctcatatgattcagtgaaagatgttctggtaccagactatggaatgagtaacctaacagcttgtaattggtgccatggcacatctgtgcgaaaggtcagaggagatgggtctattgtgtatttggatggagacagaactaattctaggtccactggtggcaaatgtggttgtggattcaaacacttttgggatggtaaggagtatgacaatctaccagaagctttccctattactttagaatggggtggaagagtggtcagagaaacagtatattggttccagtatgaaagtgattcatctttgaatagtaatgtttacgatgttgcaatgaaacttgttaccaagcactttccaggtgaatttgggagtgaaatcctagttcagaaagttgtccacactatattgcatcagactgccaaaaagaatcccgatgattatactcctgtaaatatagatggtgctcacgcccaaagagttggagatgttcaaggacaagaatcagagtctcagctcccaactaaaattattcttactggacagaaaacaaaaactttgcacaaggaggagttaaacatgagtaaaactgaaagaactattcaacagaatattacggaacaggcttctgtaatgcagaaacggaaaacagagaagttaaaacaagaacaaaaagggcaacccaggactgtttctcccagtaccattcgtgatggtccatcctctgcacctgctacacctaccaaggctccctattcaccgacaacttctaaggagaagaagatccgaatcacaactaatgatggacgacagtccatggttacccttaagtcttcaacaaccttttttgaacttcaggaaagtatagccagagaattcaacattcctccatatttacagtgtattcgatacgggtttcctcctaaagagttaatgccaccacaggcaggaatggaaaaggaaccagttcctttacagcatggcgacagaattacaatagaaattctaaaaagtaaagctgaaggtggtcagtctgctgcagcacactcagcccacactgtgaaacaagaagatattgctgttactggtaaactgtcatctaaggaacttcaggagcaagctgaaaaagaaatgtactccttgtgtcttttagcaacattaatgggagaagatgtatggtcttatgcaaagggacttcctcacatgtttcagcagggtggtgtattctacagt

SEQ ID NO: 42 - protein, 902 aa, Homo sapiens
  REGION 1..902: note = VCIP135 Human UBX-Like
SSLASAAASGGLLKRRDRRILSGSCPDPKCQARLFFPASGSVSIECTECGQRHEQQQLLGVEEVTDPDVVLHNLLRNALLGVTGAPKKNTELVKVMGLSNYHCKLLSPILARYGMDKQTGRAKLLRDMNQGELFDCALLGDRAFLIEPEHVNTVGYGKDRSGSLLYLHDTLEDIKRANKSQECLIPVHVDGDGHCLVHAVSRALVGRELFWHALRENLKQHFQQHLARYQALFHDFIDAAEWEDIINECDPLFVPPEGVPLGLRNIHIFGLANVLHRPIILLDSLSGMRSSGDYSATFLPGLIPAEKCTGKDGHLNKPICIAWSSSGRNHYIPLVGIKGAALPKLPMNLLPKAWGVPQDLIKKYIKLEEDGGCVIGGDRSLQDKYLLRLVAAMEEVFMDKHGIHPSLVADVHQYFYRRTGVIGVQPEEVTAAAKKAVMDNRLHKCLLCGALSELHVPPEWLAPGGKLYNLAKSTHGQLRTDKNYSFPLNNLVCSYDSVKDVLVPDYGMSNLTACNWCHGTSVRKVRGDGSIVYLDGDRTNSRSTGGKCGCGFKHFWDGKEYDNLPEAFPITLEWGGRVVRETVYWFQYESDSSLNSNVYDVAMKLVTKHFPGEFGSEILVQKVVHTILHQTAKKNPDDYTPVNIDGAHAQRVGDVQGQESESQLPTKIILTGQKTKTLHKEELNMSKTERTIQQNITEQASVMQKRKTEKLKQEQKGQPRTVSPSTIRDGPSSAPATPTKAPYSPTTSKEKKIRITTNDGRQSMVTLKSSTTFFELQESIAREFNIPPYLQCIRYGFPPKELMPPQAGMEKEPVPLQHGDRITIEILKSKAEGGQSAAAHSAHTVKQEDIAVTGKLSSKELQEQAEKEMYSLCLLATLMGEDVWSYAKGLPHMFQQGGVFYS

SEQ ID NO: 43 - DNA, 849 bp, Homo sapiens
  misc_feature 1..849: note = YOD1 Human UBX-Like
  source 1..849: mol_type = other DNA
cggaccgacacgatgtggcggctccgctgcaaggccaaggacggcacccatgttttgcaggggctgtccagccggacccgggtgcgggaactccagggccaaattgccgccatcaccgggatcgcccccggcggtcagcgaatcctcgtcggataccctcccgagtgcctggatctcagcaatggggataccattctggaagacttgcccatccaatctggtgacatgctgatcattgaagaagaccaaaccaggcccagaagttcacctgcatttactaaacgtggtgcttctagttacgtcagggaaactttgcctgtgcttaccagaaccgtggtcccagcagacaactcttgcctctttactagtgtgtactatgtcgtcgaaggaggagtcttgaatccagcttgtgcccctgagatgagacgcctcatagcacaaattgtagcaagcgatccagacttctatagtgaggcaatactgggaaaaacaaatcaagagtactgtgactggatcaaaagggatgacacttggggaggagcaatagagatatcgattttgtccaagttttaccaatgtgaaatatgtgtagtggatacacagacagtaagaattgatcgttttggggaagatgcaggatataccaaaagggttctgcttatttatgatggcatccactatgatccacttcagcgtaacttccctgatccagatacacctcctctgaccattttctcctctaatgatgatattgttcttgtacaagcactggaattagcagatgaagctagaagaaggagacagtttactgatgtcaaccgcttcaccctgagatgcatggtatgtcag

SEQ ID NO: 44 - protein, 282 aa, Homo sapiens
  REGION 1..282: note = YOD1 Human UBX-Like
RTDTMWRLRCKAKDGTHVLQGLSSRTRVRELQGQIAAITGIAPGGQRILVGYPPECLDLSNGDTILEDLPIQSGDMLIIEEDQTRPRSSPAFTKRGASSYVRETLPVLTRTVVPADNSCLFTSVYYVVEGGVLNPACAPEMRRLIAQIVASDPDFYSEAILGKTNQEYCDWIKRDDTWGGAIEISILSKFYQCEICVVDTQTVRIDRFGEDAGYTKRVLLIYDGIHYDPLQRNFPDPDTPPLTIFSSNDDIVLVQALELADEARRRRQFTDVNRFTLRCMVC

SEQ ID NO: 45 - DNA, 1506 bp, Homo sapiens
  misc_feature 1..1506: note = NPL4 Human UBX-Like
  source 1..1506: mol_type = other DNA
atggccgagagcatcataattcgtgtccagtccccggatggagtgaagcggatcacagcaacaaagagagaaacagcagcaacatttttgaaaaaggttgcaaaggagtttggcttccaaaataatggcttctcggtttacatcaatagaaacaagaccggagagataacagcctcctccaacaaatccctcaacttgctaaaaatcaagcatggcgatttgttgttcctgtttccctcgagccttgctgggccctcatctgaaatggagacgtcagttccaccgggcttcaaagtctttggcgctcccaacgtggtggaggatgagattgatcagtacctcagcaaacaggacgggaagatttacagaagccgagacccacagctatgccgccacggccctttggggaaatgcgtgcactgcgtccctctagagccattcgatgaggactatctaaaccatctcgagcctcccgtgaagcacatgtccttccacgcctacatccggaagctgactggaggggctgacaaggggaagtttgttgccctggagaacatcagctgcaagattaagtcagggtgcgaggggcacctcccgtggccgaatggcatctgtactaagtgccagccgagcgccatcacgctgaacagacagaagtacaggcatgtggacaatatcatgtttgagaatcacaccgtcgctgaccgctttcttgacttctggagaaagacagggaaccagcattttgggtacttatacggacggtacacggagcacaaagacattccccttggcatcagggctgaagtggctgcgatttatgagccacctcagattggtacacagaacagcttggagcttcttgaggatccaaaagctgaagtggtcgatgaaattgctgccaaacttggcctgcggaaggttggctggatatttacagacctcgtctcagaagatacccgaaagggtaccgtccgctacagtcgaaataaggacacctatttcctaagttcagaagagtgcatcactgcaggagacttccagaacaagcatcccaacatgtgccggctctctccagacggacattttggatccaagtttgttactgcagtggctacaggtggtcctgacaaccaagtccactttgaagggtaccaggtgtccaatcagtgtatggcactggtccgtgatgagtgtttgctgccatgcaaggacgccccggagcttggctacgccaaggagtctagcagtgagcagtacgtgcctgatgtgttttataaggacgtagacaagtttggcaacgagatcacccagctggcccggcccctgcctgtggagtatctcatcatagacatcacaacaactttccccaaggatccagtttacactttttctatttcgcaaaatccatttcctattgaaaaccgggatgtattgggtgagacacaggacttccatagcttggccacctatttgtctcagaatacctcatctgtgttcttggat

SEQ ID NO: 46 - protein, 501 aa, Homo sapiens
  REGION 1..501: note = NPL4 Human UBX-Like
MAESIIIRVQSPDGVKRITATKRETAATFLKKVAKEFGFQNNGFSVYINRNKTGEITASSNKSLNLLKIKHGDLLFLFPSSLAGPSSEMETSVPPGFKVFGAPNVVEDEIDQYLSKQDGKIYRSRDPQLCRHGPLGKCVHCVPLEPFDEDYLNHLEPPVKHMSFHAYIRKLTGGADKGKFVALENISCKIKSGCEGHLPWPNGICTKCQPSAITLNRQKYRHVDNIMFENHTVADRFLDFWRKTGNQHFGYLYGRYTEHKDIPLGIRAEVAAIYEPPQIGTQNSLELLEDPKAEVVDEIAAKLGLRKVGWIFTDLVSEDTRKGTVRYSRNKDTYFLSSEECITAGDFQNKHPNMCRLSPDGHFGSKFVTAVATGGPDNQVHFEGYQVSNQCMALVRDECLLPCKDAPELGYAKESSSEQYVPDVFYKDVDKFGNEITQLARPLPVEYLIIDITTTFPKDPVYTFSISQNPFPIENRDVLGETQDFHSLATYLSQNTSSVFL

(19) United States
(12) Patent Application Publication
(10) Pub. No.: US 2025/0223341 A1
(43) Pub. Date: Jul. 10, 2025

(54) P97-BASE PROTAC ANTIBODY CONJUGATES AND METHODS OF USE

(71) Applicant: UNIVERSIDAD AUSTRAL DE CHILE, Valdivia (CL)

(72) Inventors: Alejandro ROJAS-FERNANDEZ, Valdivia (CL); Constanza SALINAS, Valdivia (CL)

(21) Appl. No.: 18/853,351

(22) PCT Filed: Apr. 1, 2023
(86) PCT No.: PCT/IB2023/053286

§ 371 Date: Oct. 1, 2024

Related U.S. Application Data
(60) Provisional application No. 63/326,454, filed on Apr. 1, 2022.

(51) Int. Cl. C07K 16/18; C07K 14/47

(52) U.S. Cl. CPC C07K 16/18; C07K 14/4747; C07K 2317/22; C07K 2317/569; C07K 2317/76; C07K 2317/82; C07K 2319/01

(57) ABSTRACT

New system to facilitate the degradation of target proteins within cells, which uses a chimeric protein with a moiety of single-domain VHH antibodies fused with the UBX domain that is recognized by p97. Where the VHH binds proteins selectively and the UBX domain recruit the protein for p97-mediated degradation based of proteasome activity. Single-domain VHH antibodies, or Nanobody (Nb) can be attached to UBX directly or using a linker.

### FIELD OF INVENTION

The invention in directed to a new p97-based proteolysis targeting chimaera (PROTAC), which facilitates the degradation of specific target proteins in a unique, Ubiquitin E3 ligase independent manner.

### BACKGROUND OF THE INVENTION

The protein p97 is a member of the AAA+ family of ATPases. It is a mechanoenzyme that actively uses energy from ATP hydrolysis to promote protein unfolding and segregation to deliver to the proteasome for degradation. Adaptor proteins mediate p97 substrate recognition. These proteins directly interact with substrates or indirectly interact with them via ubiquitin-modifications to internalize substrates into p97 hexamer's lumen. The adaptor proteins interact with p97 via a specific p97 binding domain called UBX. We engineered synthetic adaptors to target p97 to specific substrates. Our system utilizes the extraordinary intracellular binding capabilities of camelid nanobodies to recognize proteins and we fused them to the UBX domain of the p97 adapter FAF1. We have developed a p97-based proteolysis targeting chimaera (PROTAC), which facilitates the degradation of specific target proteins in a unique, Ubiquitin E3 ligase independent manner.

### Ubiquitin-Proteasome System and Conventional Proteolysis Targeting Chimaera (PROTAC)

The ubiquitin-proteasome system regulates protein levels by recognizing Ubiquitin E3 ligases. The formation of ubiquitin chains on the substrates labels them for proteasome-mediated degradation. Constitutive protein degradation is a rapid negative feedback mechanism and an important stress response pathway. For instance, the transcription factor Hif1α accumulates during hypoxia and triggers the expression of several genes controlled by a promoter containing hypoxia response elements (HREs). Interestingly, under normoxia, Hif1α is downregulated by constant ubiquitin-mediated proteasomal degradation. Hif1α is rapidly hydroxylated in an oxygen-dependent manner. Hif1α hydroxylation facilitates VHL binding to adaptors of the Ubiquitin E3 ligase Cullin-2, leading to constitutive ubiquitination degradation of Hif1α. Hypoxia limits the oxygen available for Hif1α hydroxylation, leading to VHL dissociation and rapid Hif1α accumulation. This natural negative regulatory mechanism was the basis for the development of the proteolysis targeting chimaera (PROTAC) technology of invention. PROTAC comprises synthetic heterobifunctional molecules that facilitate the degradation of proteins of interest (POIs) by ubiquitinating them in a ubiquitin E3 ligase-dependent manner. PROTAC vary from small molecules to protein domains and antibodies fragments. For instance, camelid Nanobodies bind proteins intracellularly stably and robustly. Nanobodies have been fused to ubiquitin E3 ligases domains or ubiquitin E3 ligases adaptors to facilitate degradation of ectopic and endogenous protein targets. For instance, the ADprom system facilitates the fusion of Nanobodies to VHL adaptors against POIs and successfully depletes endogenous targets Fulcher L J, Macartney T, Bozatzi P, Hornberger A, Rojas-Fernandez A, Sapkota G P. An affinity-directed protein missile system for targeted proteolysis. Open Biol. 2016 October; 6 (10):160255. doi: 10.1098/rsob.160255. PMID: 27784791; PMCID: PMC5090066. Nanobodies have also been fused the Ubiquitin E3 ligases active domains directly. For instance, the antibody RING-mediated destruction system (ARMeD) uses a Ubiquitin E3 ligase RNF4 RING finger domain fused to Nanobodies. Importantly, the ARMED system is independent of the endogenous ubiquitin E3 ligase machinery Ibrahim A F M, Shen L, Tatham M H, Dickerson D, Prescott A R, Abidi N, Xirodimas D P, Hay R T. Antibody RING-Mediated Destruction of Endogenous Proteins. Mol Cell. 2020 Jul. 2; 79(1):155-166.e9. doi: 10.1016/j.molcel.2020.04.032. Epub 2020 May 25. PMID: 32454028; PMCID: PMC7332993.

### P97

In eukaryotic cells, AAA type ATPase p97 facilitate conventional ubiquitination and proteasomal-mediated degradation and the selection of substrates for proteasomal degradation van den Boom J, Meyer H. VCP/p97-Mediated Unfolding as a Principle in Protein Homeostasis and Signaling. Mol Cell. 2018 Jan. 18; 69(2):182-194. doi: 10.1016/j.molcel.2017.10.028. Epub 2017 Nov. 16. PMID: 29153394. It has been proposed that p97 facilitates protein complex disassembly via its ATP dependent segregase activity Noi K, Yamamoto D, Nishikori S, Arita-Morioka K, Kato T, Ando T, Ogura T. High-speed atomic force microscopic observation of ATP-dependent rotation of the AAA+ chaperone p97. Structure. 2013 Nov. 5; 21(11):1992-2002. doi: 10.1016/j.str.2013.08.017. Epub 2013 Sep. 19. PMID: 24055316. p97 uses ATP hydrolysis to ‘segregate’ ubiquitylated proteins from their binding partners, and its action is mediated by adapters or cofactors. The Adapters and Cofactors are divided into two groups depending on the site bind: a small group binds to the C-terminus of p97, while the larger group corresponds to cofactors that bind to the N-terminal domain either through a domain UBX (ubiquitin regulatory X)/UBXL (UBX-like domain motifs) or three linear binding motifs, named VCP-interaction motif (VIM), VBM (VCP-binding motif) and SHP (BS1, binding segment 1). The UBX domain is the most common and conserved motif among the cofactors, such as UBXN1 (SAKS1), UBXN2A (UBXD4), UBXN2B (p37), NSFL1C (p47), FAF1, FAF2 (UBXD8 or ETEA), UBXN4 (UBXD2), UBXN6 (UBXD1), UBXN7 (UBXD7), UBXN8 (Rep-8 or UBXD6), ASPSCR1 (ASPL or TUG), UBXN10 (UBXD3) and UBXN11 (SOCI or UBXD5). The UBX-L domain adopts a similar structure to the UBX domain (UBX-L containing domains such as VCIP135, YOD1, NPL4). Hirabayashi M, Inoue K, Tanaka K, Nakadate K, Ohsawa Y, Kamei Y, Popiel A H, Sinohara A, Iwamatsu A, Kimura Y, Uchiyama Y, Hori S, Kakizuka A. VCP/p97 in abnormal protein aggregates, cytoplasmic vacuoles, and cell death, phenotypes relevant to neurodegeneration. Cell Death Differ. 2001 October; 8(10):977-84. doi: 10.1038/sj.cdd.4400907. PMID: 11598795. p97 have been shown to desegregate poly-Q aggregates in vitro Ghosh D K, Roy A, Ranjan A. The ATPase VCP/p97 functions as a disaggregase against toxic Huntingtin-exon1 aggregates. FEBS Lett. 2018 August; 592(16):2680-2692. doi: 10.1002/1873-3468.13213. Epub 2018 Aug. 13. PMID: 30069866. Ubiquitin is often found inside aggregates related to neurodegenerative diseases, suggesting dysfunctional ubiquitin-mediated degradation machinery. We engineered a synthetic p97 adapter by fusing the UBX domain of the protein FAF1 (SEQ ID No 1) to camelid nanobodies to assemble a p97-based PROTAC (p97-PROTAC). This new chimaera efficiently targets proteins for segregation and proteasome-mediated degradation.

### BRIEF DESCRIPTION OF THE DRAWINGS

FIG. 1. (Top) UBD-UBX p97 Adapters Diagram of p97 presenting ubiquitinated proteins (S, substrate) to the proteasome via a UBX domain contacting adaptor; (Bottom) p97-PROTAC system, consisting of a UBX domain fused to a nanobody (Nb) that recruits substrates for p97-mediated segregation, unfolding and proteasomal mediated degradation; allows that complex p97-UBX-NB (nanobody), present to proteosome any Substrates(S), without needed of Ubiquitin.

FIG. 2. A) Hela cells were transfected with GFP-Coilin or co-transfected with UBX-Nb (GFP) to induce degradation. Protein degradation was further analyzed via Western blot; and its quantification.

B) Hela cells were transfected with GFP-Emerin or co-transfected with UBX-Nb (GFP) to induce degradation. Protein degradation was analyzed via Western blot; and its quantification.

C) Hela cells were transfected with GFP-ETV1 or co-transfected with UBX-Nb (GFP) to induce degradation. Protein degradation was analyzed via Western blot; and its quantification.

FIG. 3.—Targeting Liquid-liquid phase separation proteins by a p97-PROTAC.

A) Recruitment of the p97-PROTAC UBX-Nb(GFP) (Red) to YFP-53BP1 (green) within Liquid-liquid phase separation structures. Data were obtained with a High-content Celldiscoverer 7. UBX-Nb (GFP) was detected using its myc-tag. B) Western blot analysis YFP-53BP1 degradation by UBX-Nb (GFP) transfection in the knock-In 53BP1 U2OS cells, C) Quantification of B. Western blots were quantified and statistically analyzed using a student's t-test. P<0.05 compared to controls. n=3.

FIG. 4.—Degradation of human proteins of clinical interest with p97-PROTAC. A) Hela cells were co-transfected with a vector expressing αSynuclein mutant A53T fused to GFP (GFP-αSynuclein A53T), and an empty vector or increasing concentrations of UBX-Nb (Syn87). GFP-αSynuclein A53T degradation was determined via Western blot using an anti αSynuclein antibody. B) Quantification of A. C) Hela cells were co-transfected with a vector expressing untagged αSynuclein mutant A53T, “α-Synuclein, which is present as a small, soluble, cytosolic protein in healthy subjects, is converted to amyloid-like fibrils in neurogenerative diseases such as Parkinson's disease (PD), dementia with Lewy bodies (DLB), and multiple system atrophy (MSA)”. Cells were co-transfected with empty vector or increasing concentrations of UBX-Nb (Syn87). Untagged αSynuclein A53T degradation was determined via Western blot using an anti αSynuclein antibody. D) Quantification of C. E) We test the effect of the proteasome inhibition, using the samples YFP monomer as substrate. The YFP monomer was co-transfected with UBX-Nb (GFP) in Hela cells with DMSO or the proteasome inhibitor MG132 for 4 h. F) Quantification of E. Western blots were quantified and statistically analyzed using a student's t-test. P<0.05 compared to controls. n=3.

### DESCRIPTION OF THE INVENTION

As we have already indicated, the invention aims at a new system to facilitate the degradation of target proteins in a cell, which uses a chimeric protein with a moiety of single-domain VHH antibodies fused with the UBX domain that is recognized by p97.

Where the specificity of the VHH used as VHH mioety, gives the system specificity since it allows the chimeric molecule to join p97 and the target, which will thus be degraded by the proteasome. Single-domain VHH antibodies, or Nanobody (Nb) can be attached to UBX directly or using a linker.

For the person skilled in the art, it will be obvious that the specificity of the chimeric molecule of the invention is given by the CDRs of the VHH used, which can be chosen from any existing nanobody that has been shown to recognize the target or molecule of interest.

### Subcellular Recruitment of p97-Based PROTAC

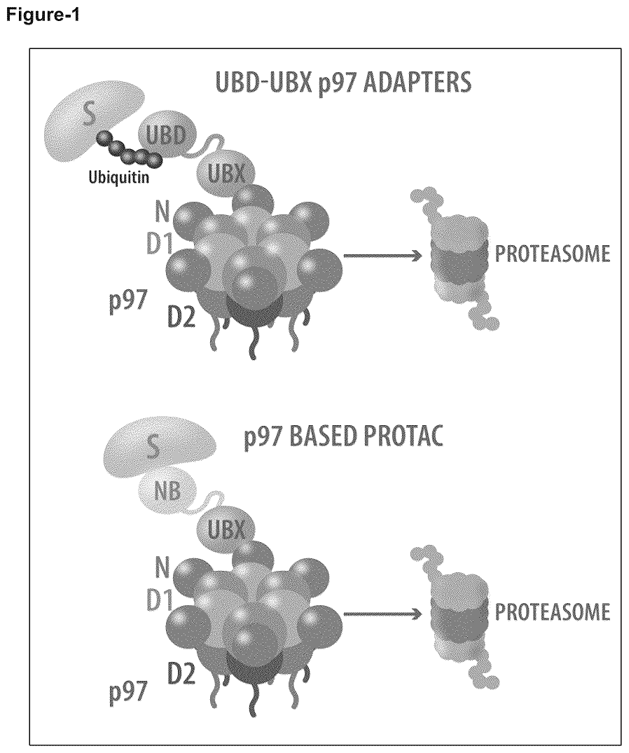

p97 adapters and cofactors specifically recognize unmodified and ubiquitin modified substrates, which enter the p97 hexamer's lumen and are sequestered for proteasomal-mediated degradation. We show that p97 adapters could be engineered to target other proteins of clinical interest and generated a new PROTAC based on p97 activity. It consists of a synthetic chimaera of the p97 adapter FAF1′s UBX domain fused to specific camelids Nanobodies (Nb), optionally by a linker (FIG. 1). (SEQ ID No 3) & (SEQ ID No 4)

So, the invention refers to:

A p97-based proteolysis targeting chimaera (PROTAC) molecule comprising a first binding domain, a second binding domain, and a linker domain, wherein:
(i) the first binding domain is configured to bind specific substrates
(ii) the second binding domain is configured to bind to the p97 AAA ATPase segregase, and
(iii) the linker domain is configured to link the first binding domain to the second binding domain under optimal conditions.

Wherein the first binding domain comprises a peptide or antibodies, and the second binding domain comprises a UBX domain.

In a preferred embodiment the second binding domain is configured to bind to the p97 AAA ATPase segregate, based on a UBX domain comprise on the mammalian proteins such as FAF1 (SEQ ID No 1) & (SEQ ID No 2) UBXN1 (SAKS1) (SEQ ID No 7) & (SEQ ID No 8), UBXN2A (UBXD4) (SEQ ID No 9) & (SEQ ID No 10), UBXN2B (p37) (SEQ ID No 11) & (SEQ ID No 12), NSFL1C (p47) (SEQ ID No 13) & (SEQ ID No 14), FAF2 (UBXD8 or ETEA) (SEQ ID No 15) & (SEQ ID No 16), UBXN4 (UBXD2) (SEQ ID No 17) & (SEQ ID No 18), UBXN6 (UBXD1) (SEQ ID No 19) & (SEQ ID No 20), UBXN7 (UBXD7) (SEQ ID No 21) & (SEQ ID No 22), UBXN8 (Rep-8 or UBXD6) (SEQ ID No 23) & (SEQ ID No 24), ASPSCR1 (ASPL or TUG) (SEQ ID No 25) & (SEQ ID No 26), UBXN10 (UBXD3) (SEQ ID No 27) & (SEQ ID No 28), UBXN11 (SOCI or UBXD5) (SEQ ID No 29) & (SEQ ID No 30), the p97 orthologous adaptor proteins of insects of the species Aedes aegypti orthologous to UBXN1. (SEQ ID No 31) & (SEQ ID No 32), orthologous to NSFL1C. (SEQ ID No 33) & (SEQ ID No 34), orthologous to FAF1 (SEQ ID No 35) & (SEQ ID No 36), orthologous to UBXN6. (SEQ ID No 37) & (SEQ ID No 38), orthologous to ASPSCR1 (SEQ ID No 39) & (SEQ ID No 40), NSFL1C and the UBX like containing proteins VCIP135 (SEQ ID No 41) & (SEQ ID No 42), YOD1 (SEQ ID No 43) & (SEQ ID No 44), and NPL4 (SEQ ID No 45) & (SEQ ID No 46).

Finally, when it is present, the linker domain comprises a standard polypeptide flexible sequences, wherein the linker domain covalently links the first binding domain to the second binding domain, taking substrates on p97 vicinity.

The target protein binding moiety based on single-domain VHH antibodies, or the first binding domain, comprise the moiety of binding to the target of an single-domain VHH antibodies, including the CDRs of the VHH. To the expert in the art would be obvious that VHH moiety could be in different forms, as long as it maintains its target binding capacity. For example, the target protein binding moiety based on single-domain VHH antibodies, could be a nanobody, a bispecific nanobody, a modified nanobody, as a nanobody humanized.

As it had said, the target protein binding moiety based on single-domain VHH antibodies, is fused directly to the UBX domain or through a peptide linker.

The UBX domain allows bind to the p97 AAA ATPase segregase, so it recruits the proteasome and mediated proteasome mediate degradation.

So, the chimeric molecule according the invention promote protein unfolding and segregation; wherein the target protein binding moiety binds a protein to be degraded.

In a preferred embodiment of invention, the protein to be degraded, or target, is an intracellular protein, and it is degraded by the ubiquitin-proteasome system.

In another specific embodiment of invention, the target protein binding moiety based on single-domain VHH antibodies is a bi-specific nanobody, and it has specificity or affinity for, or binds to, an extracellular protein and an intracellular protein.

Additionally, the chimeric molecule of invention further comprises a nuclear localization signal.

For the expert in the art, would be obvious that this new chimeric molecule have many different applications and uses, depending of the affinity of the binding moiety based on single-domain VHH antibodies, or the first binding domain, and of the UBX domain utilized.

For example, the chimeric molecule of invention is useful to degrading a target protein into a cell. So, it would be use in medicine or for use as a medicament; for example in treating cancer, or in treating a neurodegenerative disorder, or on conferring intrinsic immune properties to plants and/or animals

Specifically, the chimeric molecule of invention is useful to target pathogens; and can be used for to prevent pathogens infection by the development of transgenic animals and plants.

In a preferred embodiment the chimeric molecule of invention is useful for to prevent pathogens infection by the application of RNA vaccines.

In another embodiment the chimeric molecule of invention is useful for application in mosquitoes, especially for use for to prevent mosquitoes to transmitted viral diseases.

The invention also relates to a method of degrading a protein, said method comprising contacting a protein to be degraded with a chimeric molecule of invention into a cell, wherein said method comprising contacting a cell expressing the protein to be degraded with a chimeric molecule of invention which binds that protein through its VHH moiety.

The invention also refers to a any nucleic acid encoding a chimeric molecule of invention, including modified RNA for vaccines.

In order to illustrate the invention chimeric molecules according to the invention were synthetized:

In a first example, see the chimeric molecule of SEQ ID No 4, useful to binding to the green fluorescent protein (GFP) and its DNA sequence, SEQ ID No 3, for its expression in a plasmid or vector.

In a second example, see the chimeric molecule of SEQ ID No 6, useful to binding to Alpha Synuclein, and its DNA sequence, SEQ ID No 5, for its expression in a plasmid or vector. This is explained in detail in the examples below.

### EXAMPLES

### Example 1. Construction of a Chimeric Molecule According to the Invention

The inventors select from their own libraries, a Nanobody against GFP, and used it to obtain a chimeric molecule according the invention, “GFP PROTAC” or p97-PROTAC Ubx-Nb^{(GFP)}.

The inventors synthesized the DNA sequence, SEQ ID No 3, which comprises coding sequencers for a tag moiety, Myc TAG, Nanobody against GFP moiety, Nb^{(GFP)}, a linker moiety, and the FAF1 UBX domain moiety. This sequence was cloned in an expression vector, expressed and recover. The protein sequence of this chimeric molecule Ubx-Nb^{(GFP)} is on SEQ ID No 4, so this chimeric molecule, is useful to binding GFP, and its DNA sequence, SEQ ID No 3, for its expression in a plasmid or vector.

### Example 2. Function Test

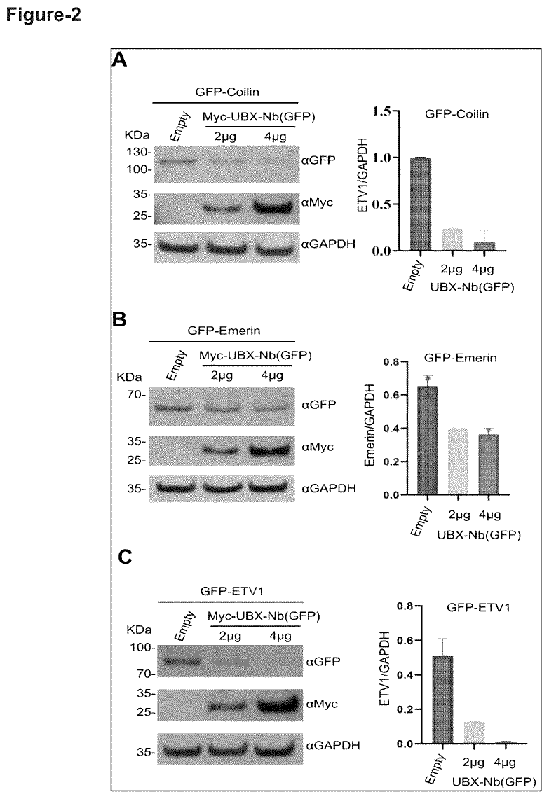

We tested the recruitment of Ubx-Nb^{(GFP)} obtain in Example 1 to a group of differentially located targets:
i) GFP alone (cyto and nucleoplasmic),
ii) GFP-Coilin (Nuclear and Cajal Bodies),
iii) GFP-Emerin (Nucleus outer membrane), and
iv) GFP-ETV1 (Nuclear) in HeLa cells.
   A) Hela cells were transfected with GFP-Coilin or co-transfected with UBX-Nb (GFP) to induce degradation. Protein degradation was further analyzed via Western blot; and its quantification is showing in FIG. 2A. In the graphic we can appreciate than even in the minimum amount, of 2 μg, the Ubx-Nb^{(GFP)}, the concentration of GFP decrease about 70%.
   B) Hela cells were transfected with GFP-Emerin or co-transfected with UBX-Nb (GFP) to induce degradation. Protein degradation was analyzed via Western blot; and its quantification is showing in FIG. 2B. The results shown a decreased GFP concentration by about 30% in the presence of 2 μg, the Ubx-Nb(GFP).
   C) Hela cells were transfected with GFP-ETV1 or co-transfected with UBX-Nb (GFP) to induce degradation. Protein degradation was analyzed via Western blot, and its quantification is showing in FIG. 2C. Newly, we can appreciate a decreased GFP concentration by about 70% or more in presence of 2 μg, the Ubx-Nb(GFP)

Consequently, the p97-based Protac Ubx-Nb^{(GFP)} efficiently recognized and sequestered GFP-fused proteins in all cellular locations. Additionally, we examined the effect of the Ubx-Nb^{(GFP)} expression on GFP-Coilin, GFP-Emerin and GFP-ETV1 protein levels. We observed a significant reduction in the levels of all GFP target proteins, indicating the p97-PROTAC-Ubx-Nb^{(GFP)} efficiently facilitates the degradation of target proteins (FIG. 2).

Therefore, p97-PROTAC is an alternative to conventional ubiquitin ligase-based PROTACs and might enhance the degradation of large protein complexes and toxic aggregates via its triple ATPase segregate activity.

### Example 3. Specific Degradation of Protein Within Liquid-Liquid Phase Separation Structures

We generated a knock-in cell line by endogenously fusing a yellow fluorescent protein to the N terminal of the 53BP1 using a CAS9 D10A nickase system to reduce potential off-target effects. We generated two specific gRNAs to target the 5′ UTR directly before the start codon of 53BP1. We transfected U2OS T Rex cells modified to produce CAS9 D10A nickase in a Doxycycline inducible manner with the sense and the antisense gRNA. Additionally, we engineered a synthetic vector with two homologue flanking regions around the cleavage site we inserted into the VFP cDNA. We mutated the gRNA recognition sequences in the synthetic vector to prevent its cleavage by the gRNA/Cas9 complex. Finally, we transfected the U2OS cells with the two gRNAs and the donor vector carrying the modified YFP cDNA and induced Cas9 D10A nickase expression 24 h after the transfection via Doxycycline.

We isolated green cells by cell sorting, and fluorescence microscopy examined the YFP-53BP1 Knock-In (KI) clones. DNA damage during DNA replication causes 53BP1 accumulation at specific foci in G1, and the YFP-53BP1 (KI) cell line fully recapitulated 53BP1 accumulation at these sites. G1 foci in the two daughter cells are symmetrical and inherited at the same chromatin position.

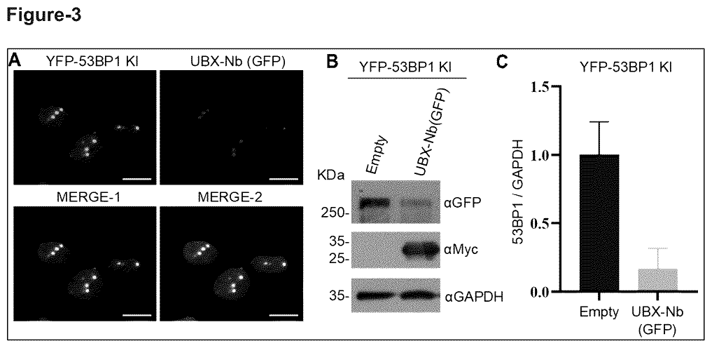

The endogenous 53BP1 promoter controls YFP-53BP1 KI fusion, and its expression and location replicated the endogenous membrane-less organelles organized by liquid-liquid phase separation (LLPS). We used these YFP-53BP1 KI cells to study p97-PROTAC Ubx-Nb ^{(GFP)} recruitment to LLPS and found the Ubx-Nb^{(GFP)} construct was successfully recruited to the DNA damage foci labelled by 53BP1 accumulation (FIG. 3A).

Finally, we also examined how Ubx-Nb^{(GFP)} affected the expression of YFP-53BP1. We observed a strong reduction of 53BP1 protein levels, suggesting that p97-PROTAC specifically catalyzes protein degradation within LLPS. We found that this new and unique p97-based PROTAC efficiently triggers the specific degradation of protein 53BP1 rom LLPS (FIG. 3B & 3C).

### Example 4. Second Chimeric Molecule According to the Invention

p97 is endogenously expressed in tissues affected in neurodegenerative diseases. We tested two aggregate models of clinical interest for p97-PROTAC-mediated degradation, such as the α-Synuclein A53T mutant. We generated another p97-PROTAC by replacing the anti-GFP nanobody with a specific α-Synuclein nanobody (NbSyn87) as a potential therapeutic measure for the reduction of α-Synuclein aggregates in neurodegenerative diseases.

Likewise, cells were transfected with a GFP fusion of the α-Synuclein A53T mutant together with an empty vector or with increasing concentrations of the anti α-Synuclein p97-PROTAC Ubx-Nb (Syn87).

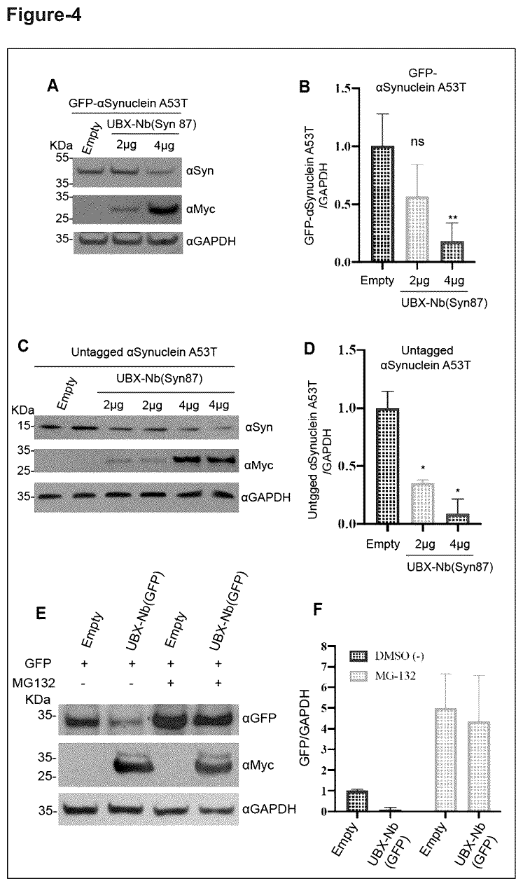

Again, we observed efficient degradation of the α-Synuclein A53T p97-PROTAC (FIG. 4A & 4B). We then replace the Nanobody against GFP by a Nanobody against alpha Synuclein Syn87and construct a similar chimera UBX-Nb^{(Syn87)} (figure C and D). Thus, we demonstrated that the nanobody component on the p97-PROTAC is exchangeable and that the p97-PROTAC system is suitable for degrading human proteins of clinical interest.

We also demonstrate that the protein levels can be rescue when cells are treated with Proteasome inhibitors such as MG132, thus the p97-PROTAC facilitated proteasome mediated degradation.

1- A chimeric molecule for specific degradation of proteins wherein the chimeric molecule comprises:
a. a target protein binding moiety based on a single-domain VHH antibody fused to
b. a UBX domain present on a p97 adaptor having at least a 90% identity with aminoacidic sequence according to: SEQ ID 2; SEQ ID 8, SEQ ID 10, SEQ ID 12, SEQ ID 14, SEQ ID 16, SEQ ID 18, SEQ ID 20, SEQ ID 22, SEQ ID 24, SEQ ID 26, SEQ ID 28, SEQ ID 30, SEQ ID 34, SEQ ID 36, SEQ ID 38, SEQ ID 40, SEQ ID 42, SEQ ID 44 or SEQ ID 46, wherein said UBX domain binds to p97.

2- The chimeric molecule of claim 1 wherein the UBX domain is encoded by a nucleotide sequence having at least a 90% identity to SEQ ID No 1, SEQ ID 7, SEQ ID 9, SEQ ID 11, SEQ ID 13, SEQ ID 15, SEQ ID 17, SEQ ID 19, SEQ ID 21, SEQ ID 23, SEQ ID 25, SEQ ID 27, SEQ ID 29, SEQ ID 33, SEQ ID 35, SEQ ID 37, SEQ ID 39, SEQ ID 41, SEQ ID 43 or SEQ ID 45.

3- The chimeric molecule of claim 1 wherein the target protein binding moiety based on a single-domain VHH antibody, comprises the target-binding moiety of a single-domain VHH antibody, including the CDRs of the VHH.

4- The chimeric molecule of claim 3 wherein the target protein binding moiety based on Alpacas VHH, is a bispecific antibody.

5- The chimeric molecule of claim 3 wherein the target protein binding moiety based on Alpacas VHH is a antibody humanized.

6- The chimeric molecule of claim 1 wherein the target protein binding moiety based on Alpacas VHH, is fused directly to the UBX domain.

7- The chimeric molecule of claim 1 wherein the target protein binding moiety based on Alpacas VHH, is fused to the UBX domain through a peptide linker.

8- The molecule of claim 1, wherein the p97 binding domain and the target protein binding moiety promote protein unfolding and segregation.

9- The molecule of claim 3, wherein the target protein binding moiety binds a protein to be degraded.

10- The molecule of claim 9, wherein the protein to be degraded is an intracellular protein.

11- The molecule of claim 4, wherein the bi-specific nanobody has specificity or affinity for, or binds to, an extracellular protein and an intracellular protein.

12- The molecule of claim 1, wherein the molecule further comprises a nuclear localization signal.

13- The molecule of claim 1 for use as a medicament.

14- The molecule of claim 1 for use in the treatment of cancer.

15- The molecule of claim 1 for use in the treatment of a neurodegenerative disorder.

16- A method of degrading a cellular protein, said method comprising contacting a cell expressing the protein to be degraded with a molecule of claim 1.

17- A nucleic acid encoding a molecule of claim 1, including modified RNA for vaccines.

18- The molecule of claim 1, wherein bind GFP and have the SEQ ID 4 and/or is encoded by the SEQ ID 3.

19- The molecule of claim 1, wherein bind Alfa Synucleina 87 and have the SEQ ID 6 and/or is encoded by the SEQ ID 5.
